(19) United States
(12) Patent Application Publication
(10) Pub. No.: US 2022/0218918 A1
(43) Pub. Date: Jul. 14, 2022

(54) ARM SUPPORT FOR INTRAVENOUS MEDICAL TREATMENTS

(71) Applicant: Maya DiMauro, Hamden, CT (US)

(72) Inventor: Maya DiMauro, Hamden, CT (US)

(21) Appl. No.: 17/145,506

(22) Filed: Jan. 11, 2021

(51) Int. Cl. A61M 5/52

(52) U.S. Cl. CPC A61M 5/52; A61M 2210/083; A61M 2205/59; A61M 2205/0205

(57) ABSTRACT

An arm support for intravenous medical treatments includes an arm support portion having a first surface and a second surface that defines a rigid support receiving zone. The first surface includes a cushioned section. At least one strap member is secured to the arm support portion. A rigid support member is positioned at the second surface at the rigid support receiving zone.

### BACKGROUND

Various medical treatments require a patient to sit in a chair or lay in a bed to receive medicine intravenously. Often times, the treatment can take hours and, when sitting in a chair, be quite uncomfortable. Prior to treatment, a patient's arm is secured to a rigid board. The board prevents bending at, for example, an elbow. The board may be free floating, e.g., the patient may move his or her arm, or may be secured to a structure such as a support provided on a chair.

If a free-floating rigid support is used, patients may rest their arm on an arm rest of the chair. In some cases, the patent is given a pillow upon which to rest his or her arm. While soft, the pillow may take up space and, left unsecured, fall to the floor. Without some form of cushion, the treatment may become more uncomfortable than necessary and, for prolonged treatments, may become very uncomfortable. Accordingly, the art would appreciate a system for rigidly supporting a patient's arm while at the same time providing comfort for prolonged intravenous treatments.

### SUMMARY

Disclosed is an arm support for intravenous medical treatments including an arm support portion having a first surface and a second surface that defines a rigid support receiving zone. The first surface includes a cushioned section. At least one strap member is secured to the arm support portion. A rigid support member is positioned at the second surface at the rigid support receiving zone.

### BRIEF DESCRIPTION OF THE DRAWINGS

The following descriptions should not be considered limiting in any way. With reference to the accompanying drawings, like elements are numbered alike:

FIG. 1 depicts an arm support for intravenous medical treatment positioned on an arm, in accordance with an exemplary aspect;

FIG. 2 depicts a rigid support member being installed into a cushioned arm support portion of the arm support for intravenous medical treatment of FIG. 1, in accordance with an exemplary aspect; and

FIG. 3 depicts the arm support for intravenous medical treatment preparing to be mounted in an arm, in accordance with an exemplary aspect.

### DETAILED DESCRIPTION

A detailed description of one or more embodiments of the disclosed apparatus and method are presented herein by way of exemplification and not limitation with reference to the Figures.

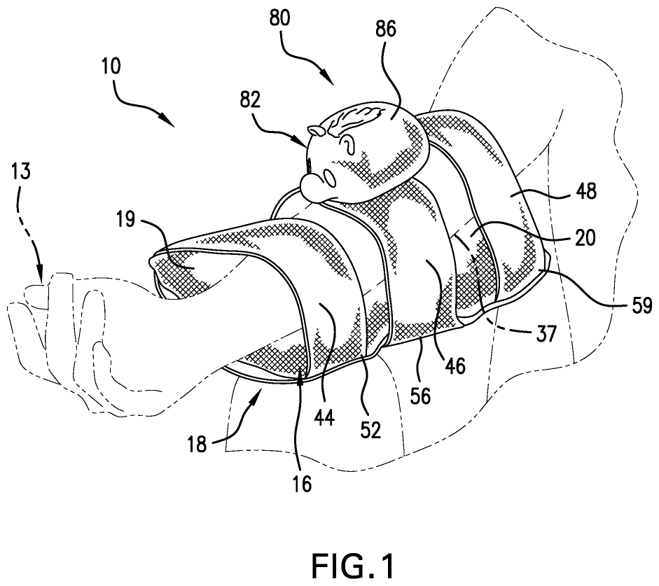

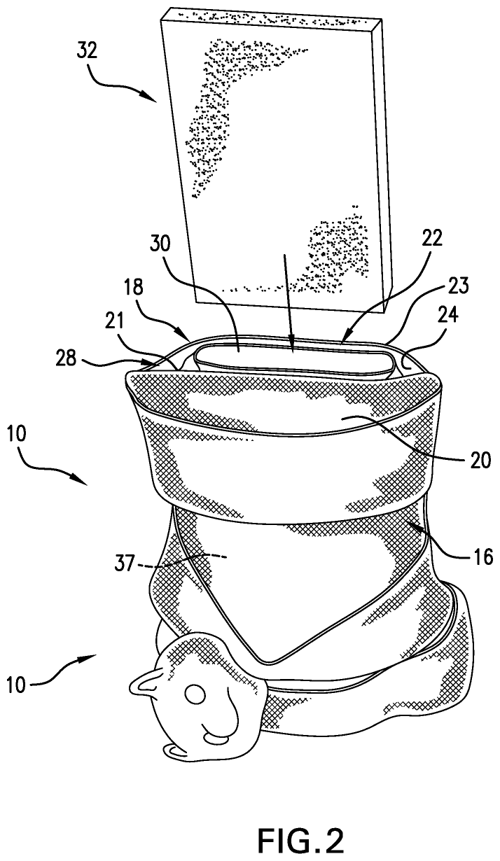

An arm support for intravenous medical treatment is indicated generally at 10 in FIG. 1 positioned on an arm 13. Arm support 10 includes a first surface 16 that may be in contact with arm 13 and a second surface 18. As shown in FIG. 2, first surface 16 may be defined by a first element 19 having a first outer surface 20 and a first inner surface 21. Second surface 18 may be formed from a second element 22 that is joined to first surface 16 and includes a second outer surface 23 and a second inner surface 24. First inner surface 21 is separated from second inner surface 24 to define a pocket 28 that may serve as a rigid support receiving zone (not separately labeled). A sleeve 30 may be arranged in pocket 28. Sleeve 30 may be a separate component or may be part of arm support 10.

In an embodiment, a rigid support member 32 may be positioned in sleeve 30. In such a case, sleeve 30 defines the rigid support receiving zone. Rigid support member 32 prevents arm 13 from bending during treatment. By inserting rigid support member 32 in sleeve 30 a user's experience with arm support 10 may be enhanced particularly through the use of cushioning as will be detailed herein. Rigid support member 32 may be formed from a variety of materials including a strictly rigid materials, as well as malleable materials that can conform to arm 13.

Arm support 10 may be formed from an anti-microbial material that reduces germ transfer between users. As an alternative, arm support 10 may be treated with an anti-microbial treatment or fluid. As noted herein, first surface 16 supports arm 13. As such, first surface 16 may have a cushioned section. More specifically, an amount of padding 37 may be present within first surface 16. Alternatively, the amount of padding 37 may be positioned in pocket 28 on one side of sleeve 30 or padding 37 may be arranged entirely about sleeve 30.

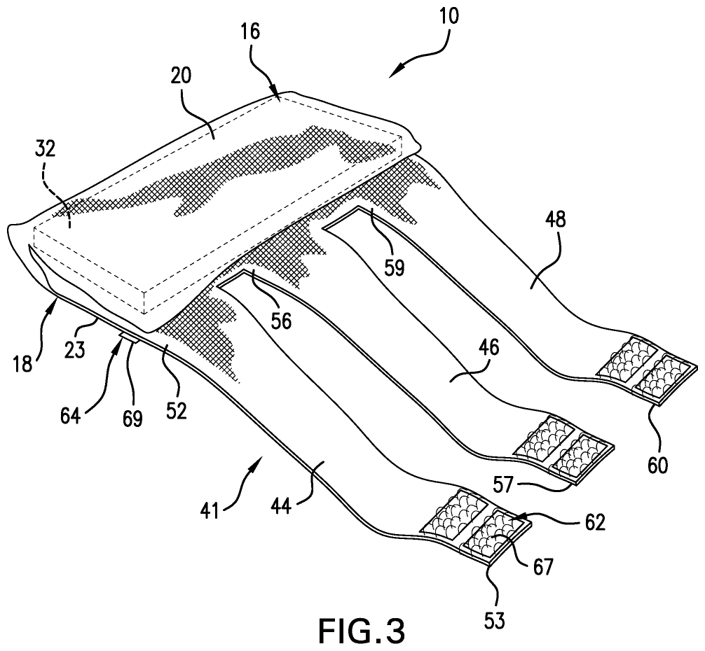

In an embodiment, arm support 10 includes one or more strap members 41. Strap members 41 are employed to secure arm support 10 to arm 13 prior to treatment. In the embodiment shown, arm support 10 includes three strap members including a first strap member 44, a second strap member 46, and a third strap member 48. Each strap member 44, 46, and 48 may encircle arm support 10 and arm 13. Strap member 44 includes a first end 52 secured to arm support 10 and a second end 53. Strap member 46 includes a first end 56 secured to arm support 10 and a second end 57. Strap member 48 includes a first end 59 secured to arm support 10 and a second end 60. Each second end 53, 57, and 60 includes a first fastener portion such as indicated at 62 in FIG. 3. A second fastener portion 64 may be disposed on second outer surface 23 of second surface 18. First fastener portion 62 may take the form of a loop fastener portion 67 while second fastener portion 64 may take the form of a hook fastener portion 69 that is configured to interconnect with loop fastener portion 67 to form a hook and loop fastener (not separately labeled).

At this point, it should be understood that while shown as extending entirely about arm support 10 in one direction, each strap member 44, 46, and 48 may pass through a loop or ring (not shown) and return about arm support 10 in a second direction. The loop may provide leverage allowing arm support to be cinched to arm 13. In other embodiments, each strap member 44, 46, and 48 may be formed from two sections that join one to another securing arm support 10 to arm 13.

In an embodiment, arm support 10 may include a distraction element 80 detachably mounted to, for example, strap member 46. Distraction element 80 may take the form of a toy 82 such as a plush toy 86 that may provide amusement for children to help pass the time during treatment. It should however be understood that toy 82 can take on a variety of forms including mechanical toys, electrical toys, interactive toys and games that may help a patient pass the time during intravenous treatment.

At this point, it should be appreciated that the exemplary embodiments define an arm support that enhances a patient's experience during intravenous drug and/or other treatment. The arm support not only prevents bending of the arm but is also cushioned so as to provide comfort without the need for bulky pillows and the like. In addition, the arm support may be provided with detachable and interchangeable distraction elements that may help young patients focus on something other than the treatment itself.

The use of the terms “a” and “an” and “the” and similar referents in the context of describing the invention (especially in the context of the following claims) are to be construed to cover both the singular and the plural, unless otherwise indicated herein or clearly contradicted by context. Further, it should be noted that the terms “first,” “second,” and the like herein do not denote any order, quantity, or importance, but rather are used to distinguish one element from another. The terms “about” and “substantially” are intended to include the degree of error associated with measurement of the particular quantity based upon the equipment available at the time of filing the application. For example, “about” and/or “substantially” can include a range of 8% or 5%, or 2% of a given value.

While the invention has been described with reference to an exemplary embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited.

What is claimed is:

1. An arm support for intravenous medical treatments comprising:
an arm support portion including a first surface, and a second surface that defines a rigid support receiving zone, the first surface including a cushioned section;
at least one strap member secured to the arm support portion; and
a rigid support member positioned at the second surface at the rigid support receiving zone.

2. The arm support according to claim 1, wherein the arm support portion includes a first element defining the first surface and a second element defining the second surface, secured to the first element, the first and second elements form a pocket.

3. The arm support according to claim 2, wherein the pocket defines the rigid support receiving zone.

4. The arm support according to claim 2, further comprising: a sleeve positioned in the pocket, the sleeve defining the rigid support receiving zone.

5. The arm support according to claim 2, wherein the first element includes an amount of padding.

6. The arm support according to claim 1, wherein the at least one strap member includes a first end coupled to the arm support portion and a second end having a first fastener portion.

7. The arm support according to claim 6, wherein the arm support portion includes a second fastener portion.

8. The arm support according to claim 7, wherein the first fastener portion comprises one of a hook fastener portion and a loop fastener portion of a hook and loop fastener and the second fastener portion comprises another of the hook fastener portion and the loop fastener portion of the hook and loop fastener.

9. The arm support according to claim 1, wherein the at least one strap member comprises three strap members.

10. The arm support according to claim 9, further comprising: a distraction element mounted to one of the three strap members.

11. The arm support according to claim 10, wherein the distraction element is a toy.

12. The arm support according to claim 11, wherein the toy is a plush toy.

13. The arm support according to claim 10, wherein the distraction element is detachably mounted to the one of the three strap members.

14. The arm support according to claim 1, wherein the arm support portion and the at least one strap member are formed from an anti-microbial material.

15. The arm support according to claim 1, wherein the arm support portion and the at least one strap member are impregnated with an anti-microbial fluid.
